(19) United States
(12) Patent Application Publication
(10) Pub. No.: US 2024/0345103 A1
(43) Pub. Date: Oct. 17, 2024

(54) METHOD OF DIAGNOSING AND TREATING CHRONIC KIDNEY DISEASE

(71) Applicant: PrognostX Health Inc., Pewee Valley, KY (US)

(72) Inventors: Aaron L. CARRITHERS, Louisville, KY (US); Stephen L. CARRITHERS, Pewee Valley, KY (US); Brennan M. CARRITHERS, New York, NY (US); Nicholas KEMPER, Crestwood, KY (US); Luke E. MORRISON, Crestwood, KY (US)

(21) Appl. No.: 18/630,041

(22) Filed: Apr. 9, 2024

Related U.S. Application Data
(60) Provisional application No. 63/552,960, filed on Feb. 13, 2024.
(60) Provisional application No. 63/458,526, filed on Apr. 11, 2023.

(51) Int. Cl. G01N 33/74; G01N 33/70

(52) U.S. Cl. CPC G01N 33/74; G01N 33/70; G01N 2800/34; G01N 2800/52

(57) ABSTRACT

A method of diagnosing and/or treating stage 3 or stage 4 chronic kidney disease (CKD) is provided herein, where a proUGN concentration level of 2.241 ng/ml or higher is indicative of stage 3 or stage 4 CKD. Additionally, a method of diagnosing and/or treating pre-CKD is also provided herein, where a proUGN concentration level of 2.05 ng/ml to and including 2.24 ng/ml in a biological sample is indicative of pre-CKD and that a patient is at risk of developing CKD. The methods may comprise measuring the amount of proUGN in a biological sample of the patient and/or administering a CKD-therapeutic agent and/or providing lifestyle and/or dietary modifications to the patient.

### CROSS-REFERENCE TO RELATED PATENT APPLICATIONS

This patent application claims the benefit of U.S. Provisional Patent Application No. 63/458,526 filed on 11 Apr. 2023 and U.S. Provisional Patent Application No. 63/552,960 filed on 13 Feb. 2024. The entire contents of each patent application recited above is hereby incorporated by reference.

### REFERENCE TO AN ELECTRONIC SEQUENCE LISTING

This application contains references to amino acid sequences and/or nucleic acid sequences which have been submitted concurrently herewith as the sequence listing .txt file entitled “91111-000004-US_28_Mar_2024_ST26.xml”, file size 4 KiloBytes (KB), created on 28 Mar. 2024. The aforementioned sequence listing is hereby incorporated by reference in its entirety.

### FIELD

The present invention relates to the field of medical diagnosis and treatment of chronic kidney disease (CKD) and pre-CKD.

### BACKGROUND

CKD is a consistently growing worldwide epidemic diagnosed in approximately 700 million individuals, and currently affects more than one in seven U.S. adults. The asymptomatic onset of CKD effectively disguises the irreversible harm imposed on unsuspecting patients over a period of many years, contributing to high morbidity and mortality rates, and the large financial burden it places on healthcare, which currently accounts for $40 billion per year in Medicare spending. Amongst adults clinically diagnosed with CKD, <10% of Stage 3 and <60% of Stage 4 are even cognizant of their ongoing chronic disease. The current standard of care makes it very difficult for physicians to reliably detect CKD in the earlier stages. The present diagnostic efficacy for CKD Stages 3 and 4 is low and, as a consequence, it is estimated that ˜35 million Americans are undiagnosed, primarily due to the intrinsic limitations of the current gold-standard diagnostic approach, estimated glomerular filtration rate (eGFR). Temporal criteria that require at least two sequentially low eGFR values be measured 90-150 days apart is applied to the standard-of-care in order to circumvent the low specificity of serum creatinine (sCr) levels to kidney disease; but this has also contributed to the lack of follow-up by patients secondary to this extended testing timeframe. For people with CKD, benefits of earlier intervention due to earlier and more accurate diagnosis are numerous and important to address. Thus, there is a substantial unmet clinical need for a timely, accurate, specific, and sensitive measure of CKD, especially during the early stages in development among high-risk groups.

### SUMMARY

In one embodiment, a method of treating stage 3 or stage 4 CKD is described herein. The method comprises administering a CKD-therapeutic agent to a subject in need thereof, wherein the subject has a proUGN concentration level of 2.241 ng/ml or higher in a biological sample of the subject. In some embodiments, the subject may have or was previously diagnosed with acute kidney injury, COVID-19, diabetes mellitus, obesity, and/or hypertension. A subject having a proUGN concentration level of 2.241 ng/ml or higher in a biological sample may be treated with a CKD-therapeutic agent such as a sodium-glucose cotransporter-2 (SGLT2) inhibitor; a potassium-sparing diuretic/selective mineralocorticoid-receptor antagonist; a thiazolidinedione; or an antifibrotic/pyridine. In some embodiments, the method of treatment may also include measuring a concentration level of proUGN in a biological sample of the subject.

Additionally, a method for diagnosing stage 3 or stage 4 CKD in a subject is also provided. The method comprises measuring a concentration level of proUGN in a biological sample of the subject, characterized in that a concentration level of proUGN of 2.241 ng/ml or higher indicates that the subject is positive for stage 3 or stage 4 CKD. Measuring the concentration level of proUGN in a biological sample of the subject may be performed by an immunoassay, such as an enzyme linked immunosorbent assay (ELISA), counting immunoassay (CIA), chemiluminescence immunoassay (CLIA), electrochemiluminescence immunoassay (ECLIA), radioimmunoassay (RIA), or immunoradiometric assay (IRMA). In some embodiments, the method of diagnosis may also include treating the subject with a CKD-therapeutic agent when the subject has a concentration level of proUGN of 2.241 ng/ml or higher.

In a further embodiment, a method of treating pre-CKD is also provided herein. The method comprises providing lifestyle and/or dietary modification instructions to a subject to decrease progression to CKD when the subject has a proUGN concentration level of 2.05 ng/ml to and including 2.24 ng/ml in a biological sample of the subject. In some embodiments, the method of treatment may also include measuring a concentration level of proUGN in a biological sample of the subject.

Additionally, a method of diagnosing pre-CKD in a subject is also provided herein. The method comprises measuring a concentration level of proUGN in a biological sample of the subject, wherein a concentration level of 2.05 ng/ml to and including 2.24 ng/ml indicates that the subject is positive for pre-CKD. In some embodiments, the method of diagnosis may also include providing lifestyle and/or dietary modification instructions to the subject to decrease progression to CKD when the subject has a concentration level of 2.05 ng/ml to and including 2.24 ng/ml.

### BRIEF DESCRIPTION OF THE DRAWINGS

FIG. 1 is a graphical representation of a receiver-operator curve (ROC) of specificity vs. sensitivity for eGFR-defined CKD subjects vs control subjects. The area under the curve (AUC) for the discrimination between eGFR-defined CKD and control is 0.9817 with a cut-off threshold value of 2.241 ng/ml.

FIG. 2 is a graphical representation of a normal distribution curve of serum proUGN values in male and female subjects with normal renal function included in the validation study of Example 1. These data indicate the appropriate Gaussian distribution expected in a general population and further authenticate the specificity of the ELISA immunoassay for the human prouroguanylin (proUGN) prohormone biomarker.

FIG. 3 is a Wilkinson Dot Plot of serum proUGN values in eGFR-defined CKD subjects vs. controls in the validation study of Example 1.

FIG. 4 is a Violin Plot of serum proUGN values in various CKD populations having high-risk comorbid conditions, such as acute kidney injury (AKI), congestive heart failure (CHF), diabetes mellitus (DM), and hypertension (HTN).

FIG. 5 is another Violin plot of serum proUGN values in various CKD populations having high-risk comorbid conditions, such as obesity, CHF, type 2 diabetes (T2D), and HTN.

### DETAILED DESCRIPTION

### A. Introduction

CKD is a common and irreversible condition with an increasing worldwide prevalence and is associated with significant patient morbidity, mortality, and a large financial burden to the patient and to the healthcare system. There is a substantial unmet clinical need for a timely, accurate, specific, and sensitive measure of kidney disease, especially during the early stages of development and/or among high-risk groups when the disease can be asymptomatic.

A number of biomarkers are known to be involved in CKD. For example, see Wasung M. E., et al. “Biomarkers of renal function, which and when?” Clinica chimica acta. Int J Clin Chem. (2015) 438:350-7:

| | Legend | Source | Physiological action |
|---|---|---|---|
| Plasma asymmetric | ADMA | Endothelial cells | Impaired clearance, |
| dimethylarginine | | | increased production |
| Fibroblast growth | FGF-23 | Osteocytes | Disordered mineral |
| facter-23 | | | metabolism, increased |
| | | | phosphaturia |
| Monocyte | MCP-1 | All nucleated cells, | Monocyte migration to |
| chemoattractant | | renal cells | inflammatory tissue |
| protein-1 | | | |
| Neutrophil | NGAL | Leukocytes, nephron | Released from lysosomes, |
| gelatinase- | | loop of Henle and | brush-border, cytoplasm |
| associated lipocalin | | collecting duct cells | of proximal tubule cells |
| Urinary cystatin-C | uCys C | All nucleated cells | Failure of reabsorption |
| | | | due to tubular damage |
| Liver-type fatty | L-FABP | Hepatocytes, kidney; | Released from cytoplasm |
| acid-binding | | proximal tubule cells | of damaged proximal |
| protein | | | tubule epithelial cells |
| Connective tissue | CTGF | All tissues | Excessive production of |
| growth factor | | | profibrotic growth factors |
| | | | and extracellular matrix |
| Transforming | TGF-β1 | All tissues | Anti-inflammatory |
| growth | | | cytokine, promotes renal |
| factor-β1 | | | fibrosis |
| Collagen-IV | COL-IV | Kidney, eye, skin | Main component of |
| | | | glomerular basement |
| | | | membrane |
| Plasma cystatin-C | pCys C | All nucleated cells | Impaired GFR or |
| | | | clearance |
| Podocalyxin | PCX | Podocytes | Podocyte structure defect |
| Nephrin | Nep | Podocytes | Integral component of |
| | | | podocyte slit diaphragm; |
| | | | released when damaged |

In addition to the biomarkers listed above, the prohormone prouroguanylin (proUGN) is another known biomarker for CKD and can be detected using an immunological assay, such as an enzyme linked immunosorbent assay (ELISA). See U.S. Patent Publication No. 2021/0263047, which is incorporated herein by reference in its entirety.

The inventors have discovered that a value or level of 2.241 ng/ml or higher proUGN indicates that a subject has at least stage 3 eGFR-defined CKD (as opposed to stage 1 or 2, which are diagnosed by urine-albumin-to-creatinine-ratio (UACR) and have normal eGFR's) and should begin treatment with a CKD-therapeutic agent. Thus, a single-assessment qualitative (positive/negative) threshold test for stage 3 (e.g. 3a or 3b), or stage 4 eGFR-defined CKD is described herein. In other words, a treat/no treat test for physicians to help in the diagnosis and treatment of stage 3 (e.g. 3a or 3b) or stage 4 CKD.

Additionally, a pre-CKD test is also described herein. The inventors have discovered that a range of proUGN concentration level of 2.05 ng/ml to and including 2.24 ng/ml shows that a subject has pre-CKD and is at risk of developing CKD. Thus this range can be used to diagnose and/or treat pre-CKD, for example, by providing lifestyle and/or dietary change instructions to reduce or prevent the progression to CKD.

### B. Definitions

The following definitions refer to the various terms used above and throughout the disclosure.

As used herein, singular articles such as “a” and “an” and “the” and similar referents in the context of describing the elements are to be construed to cover both the singular and the plural, unless otherwise indicated herein or clearly contradicted by context.

As used herein, the term “or” is used to mean “and/or” unless explicitly indicated to refer to alternatives only or if the alternatives are mutually exclusive.

As will be understood by one skilled in the art, for any and all purposes, all ranges disclosed herein also encompass any and all possible subranges and combinations of subranges thereof.

As used herein, chronic kidney disease or “CKD” is “eGFR-defined CKD” and defined as the presence of decreased renal function and an estimated glomerular filtration rate (eGFR) less than 60 ml/min/1.73 m², persisting for three months or more in a subject or patient.

As used herein, the term “eGFR-defined CKD” includes KDIGO stages 3a, 3b, 4, and 5, even though there are also KDIGO stages 1 and 2. See Kidney Disease Improving Global Outcomes (KDIGO), Kidney International Suppl, 3, 5-14, 2013, and Kidney Disease Outcomes Quality Initiative (KDOQI™), American J. Kidney Dis., 63, 713-735, 2014, which are both hereby incorporated by reference in their entirety. Each stage of eGFR-defined CKD is represented by the following eGFR baseline values:

GFR categories in CKD

| CFR category | GFR (ml/min/1.73 m²) | Terms |
|---|---|---|
| G1 | ≥90 | Normal or high |
| G2 | 60-89 | Mildly decreased* |
| G3a | 45-59 | Mildly to |
| | | moderately decreased |
| G3b | 30-44 | Moderately to |
| | | severely decreased |
| G4 | 15-29 | Severely decreased |
| G5 | <15 | Kidney failure |

Abbreviations: CKD, chronic kidney disease; GFR, glomerular filtration rate.
*Relative to young adult level
In the absence of evidence of kidney damage, neither GFR category G1 nor G2 fulfill the criteria for CKD.

As used herein, “pre-CKD” is defined as a physiological state of a subject where the eGFR baseline has not been established to be lower than 60 ml/min/1.73 m²—and thus cannot clinically validate chronicity of depreciated renal function or kidney disease—yet, the subject has a proUGN level that exceeds the upper limit of the 95% confidence interval reference range of the general population, but remains lower than the eGFR-defined CKD threshold of 2.241 ng/ml. In other words, the term “pre-CKD” is used for individuals with an abnormally elevated proUGN level who are at risk of developing CKD and do not meet the criteria for CKD diagnosis. These individuals have proUGN levels higher than the 95% reference interval in healthy patients (0.162-2.049 ng/ml) but are below the threshold for eGFR-CKD identification as described in this work of 2.241 ng/mL. Thus, any patient/subject/individual with a value of 2.05 ng/ml to and including 2.24 ng/mL, who are at risk of developing CKD, should be considered “pre-CKD.”

GUCA2B Gene Product: proUGN
SEQ ID NO: 1: 86-amino acid human prouroguanylin (h. GUCA2B₍₁₋₈₆₎)
SEQ ID NO: 2: N-terminal [NT] 70-amino acid pro-region, [NT]pro-GUCA2B₍₁₋₇₀₎
SEQ ID NO: 3: N-terminal [NT] 22-amino acid pro-region, [NT]pro-GUCA2B₍₂₀₋₄₁₎
SEQ ID NO: 4: C-terminal 16-amino acid, UGN₍₇₁₋₈₆₎

Guca2b (Guanylate Cyclase Activator 2B) is a gene (see NCBI human gene ID: 2981) located on chromosome 1 (localization 1p34-33, respectively) that encodes a 112-amino acid preproprotein that is proteolytically processed to generate multiple protein products, including uroguanylin (UGN₇₁₋₈₆), a member of the guanylin family of peptides and an endogenous ligand of the guanylate cyclase-C (GC-C) receptor. Binding of this peptide to its cognate receptor stimulates an increase in cyclic GMP and functions to regulate salt and water homeostasis in the intestine and kidneys. In humans, post-translational intracellular cleavage of the preproprotein reveals a 26-amino acid N-terminal signal peptide and the 86-amino acid prouroguanylin (proUGN) (h. GUCA2b₍₁₋₈₆₎ (see SEQ ID NO:1), which is then released apically into the lumen and basolaterally into the vascular space and bloodstream. Once into circulation, the prohormone is then processed near the site of the kidney receptor lining within the lumen of the nephron (as well as other sites around the body like the intestine) into the active peptide hormone C-terminal 16-amino acid, UGN₍₇₁₋₈₆₎ (see SEQ ID NO: 4), as well as the N-terminal [NT] 70-amino acid pro-region, [NT]pro-GUCA2B₍₁₋₇₀₎ (see SEQ ID NO: 2).

In one embodiment, the human GUCA2B gene product or human wild-type “proUGN” as used herein is an 86 amino acid protein having the sequence as shown in SEQ ID NO: 1. The term proUGN as used herein is intended to encompass detecting a GUCA2B gene product, such as proUGN having SEQ ID NO: 1, or a fragment thereof, or a variant thereof. In some embodiments, the proUGN tertiary structure may include a di-sulfide bond.

### Fragments

The term “proUGN” herein is intended to include fragments of proUGN, such as an “N-terminal proUGN,” which includes the “pro” portion/sequence of proUGN (SEQ ID NO: 2, amino acids 1-70) and excludes the “UGN” portion/sequence (SEQ ID NO: 4, amino acids 71-86). In some embodiments, a proUGN fragment is a functional proUGN fragment. Any fragment of SEQ ID NO: 2 that is capable of accurately measuring the level of proUGN in a circulating biological sample may be used. One skilled in the art can easily determine if a proUGN fragment is accurately measuring the level or amount of proUGN in a biological sample. For example, in some embodiments, the level or amount of an “N-terminal proUGN” fragment can be detected and used to diagnose and/or treat stage 3 or stage 4 CKD or pre-CKD as provided herein. In some embodiments, the proUGN fragment may be a polypeptide having 70%, 75%, 85%, 90%, 95%, 96%, 97%, 98%, or 99% sequence identity to SEQ ID NO: 2. In other embodiments, the proUGN fragment may be a polypeptide having 70%, 75%, 85%, 90%, 95%, 96%, 97%, 98%, or 99% sequence identity to SEQ ID NO: 3.

### Post-Translational Modifications

Additionally, the use of proUGN herein is intended to include both unmodified proUGN and post-translational modified forms of proUGN, such as phosphorylated, nitrosylated, methylated, glycated, glycosylated, isoprenylated, or amidated proUGN. Thus, the level or amount of unmodified or post-translationally modified proUGN can be used to diagnose and/or treat stage 3 or stage 4 CKD or pre-CKD as provided herein.

### Variants

Additionally, the use of proUGN herein is intended to include variants of proUGN, such as SNP variants, that are capable of measuring the level or amount of proUGN in a biological sample. One skilled in the art can easily determine if a proUGN variant is accurately measuring the level or amount of proUGN in a biological sample. For example, in some embodiments, the level or amount of a proUGN variant can be used to diagnose and/or treat stage 3 or stage 4 CKD or pre-CKD as provided herein. In some embodiments, the proUGN variant may be a polypeptide having 70%, 75%, 85%, 90%, 95%, 96%, 97%, 98%, or 99% sequence identity to SEQ ID NO: 1, 2 or 3.

Additionally, the term proUGN does not include uroguanylin.

### C. Method of Diagnosing and/or Treating Stage 3 or Stage 4 CKD

In one embodiment, a method of treating stage 3 or stage 4 CKD is described herein. The method comprises administering a CKD-therapeutic agent to a subject in need thereof, wherein the subject has a proUGN concentration level of 2.241 ng/ml or higher in a biological sample of the subject.

In some embodiments, a method of treating stage 3 CKD, including stage 3a and/or stage 3b CKD, is provided. As noted above, subjects with stage 3a have a GFR of 45-59 ml/min/1.73 m² and subjects with stage 3b have a GFR of 30-44 ml/min/1.73 m² according to KDIGO. Additionally or alternatively, a method of treating stage 4 CKD is provided. A subject with stage 4 has a GFR of 15-29 ml/min/1.73 m² according to KDIGO.

### Patient Populations

As discussed above, there is a substantial unmet clinical need for a timely, accurate, specific, and sensitive measure of CKD, especially during the early stages and in development among high-risk groups. Thus, in some embodiments the subject has acute kidney injury (AKI) about 12 months or less prior to a proUGN concentration level of 2.241 ng/ml or higher in a biological sample. For example, the subject may have recently suffered from an AKI within the past 12 months from a positive test of 2.241 ng/ml or higher proUGN and were not previously diagnosed with CKD.

Additionally or alternatively, the subject may have or have been diagnosed with COVID-19 about 12 months or less prior to a proUGN concentration level of 2.241 ng/ml or higher in the biological sample. Thus, in some embodiments the subject may be diagnosed with AKI, COVID-19, or AKI and COVID-19 within about 12 months or less prior to a proUGN concentration level of 2.241 ng/ml or higher in the biological sample.

Additionally or alternatively, the subject may have or have been diagnosed with diabetes mellitus (DM), type 2 diabetes (T2D), and/or hypertension (HTN). These two groups or patient populations having diabetes mellitus and/or hypertension account for greater than 80% of all new CKD cases each year and are at the highest risk of developing CKD. Therefore, early determination of CKD in these patient populations by determining a proUGN level of 2.241 ng/ml or higher is very beneficial.

Additionally or alternatively, the subject may have or have been diagnosed with congestive heart failure (CHF).

Additionally or alternatively, the subject may have clinically-diagnosed obesity.

Additionally or alternatively, the subject may have or have been diagnosed with drug-induced nephrotoxicity/renal dysfunction. Drug-induced nephrotoxicity can be caused by many different drugs. Non-limiting examples of such drugs and conditions caused therefrom include:
1. Lithium
   Chronic interstitial nephritis, glomerulonephritis, rhabdomyolysis
2. Acetaminophen/Aspirin
   Chronic interstitial nephritis
3. Nonsteroidal Anti-inflammatory drugs (NSAIDS)
   Acute and/or Chronic interstitial nephritis, glomerulonephritis, and altered intraglomerular hemodynamics
4. Antimicrobials (various).
   Tubular cell toxicity, crystal nephropathy, interstitial nephritis
5. Chemotherapeutics (various)
   Chronic interstitial nephritis, tubular cell toxicity, glomerulonephritis, crystal nephropathy, thrombotic microangiopathy
6. Diuretics (Loops, thiazides)
   Interstitial nephritis

### CKD-Therapeutic Agents

The inventors discovered a clinical cut-off value of 2.241 ng/ml or higher proUGN which accurately determines to treat or not treat a subject with a CKD-therapeutic agent. In other words, a subject having a level of 2.241 ng/ml or higher of proUGN in a biological sample is diagnosed with stage 3 (including 3a or 3b) or stage 4 eGFR-defined CKD and therapeutic treatment should be initiated with an appropriate CKD-therapeutic agent for stage 3 or stage 4 CKD. The CKD-therapeutic agent may be a sodium-glucose cotransporter-2 (SGLT2) inhibitor; a potassium-sparing diuretic/selective mineralocorticoid-receptor antagonist; a thiazolidinedione; or an antifibrotic/pyridine.

In one embodiment, the CKD-therapeutic agent is a SGLT2 inhibitor, such as dapagliflozin, empagliflozin or canagliflozin.

In another embodiment, the CKD-therapeutic agent is a potassium-sparing diuretic/selective mineralocorticoid-receptor antagonist, such as finerenone.

In another embodiment, the CKD-therapeutic agent is a thiazolidinedione, such as pioglitazone.

In another embodiment, the CKD-therapeutic agent is an antifibrotic/pyridine, such as pirfenidone.

In another embodiment, the CKD-therapeutic agent is a glucagon-like peptide-1 receptor (GLP-1R) agonist, such as semaglutide, liraglutide, albiglutide or dulaglutide.

In another embodiment, the CKD-therapeutic agent is a guanylate cyclase-C (GCC) agonist, such as linaclotide or plecanatide.

In another embodiment, the CKD-therapeutic agent is a soluble guanylate cyclase (sGC) stimulator, such as riociguat and vericiguat, or a sGC activator, such as runcaciguat and cinaciguat.

### Biological Sample

The method of treatment includes administering a CKD-therapeutic to a subject that has a proUGN concentration level of 2.241 ng/ml or higher in a biological sample from the subject. The proUGN level may be measured in any appropriate biological sample, such as a biological fluid sample, a biological tissue sample or an exosome.

In one embodiment, the biological sample is a circulating biological fluid sample, such as serum, blood, urine, plasma, saliva, cerebrospinal fluid, amniotic fluid, lung lavage, nasal lavage, gastrointestinal fluid, or interstitial fluid.

In another embodiment, the biological sample is a biological tissue sample, such as an organic tissue biopsy from kidney or gastrointestinal tract such as stomach, small intestine, or large intestine including but not limited to the cecum, colon, or rectum.

In another embodiment, exosomes may be used as the biological sample. For example, see Zhang, Y., et al. “Exosomes: biogenesis, biologic function and clinical potential.” Cell Biosci 9, 19 (2019); Wang, X., et al. “The updated role of exosomal proteins in the diagnosis, prognosis, and treatment of cancer.” Exp Mol Med 54, 1390-1400 (2022); and Lai, J. J., et al. “Exosome Processing and Characterization Approaches for Research and Technology Development.” Adv. Sci. 9, 2103222, (2022), which are each hereby incorporated by reference in their entirety.

### Optional Steps

In addition to administering a CKD-therapeutic agent to a subject, the method of treating may also include a measuring step wherein the subject's proUGN level in a biological sample, such as blood, urine, etc., is measured. If the subject's proUGN level is 2.241 ng/ml or higher, then CKD-therapeutic treatment is initiated. Various methods/assays for measuring proUGN in a biological sample are described below.

### Method of Diagnosis

In another embodiment, a method for diagnosing stage 3 (including 3a or 3b) or stage 4 eGFR-defined CKD in a subject is provided herein. The method comprises measuring a concentration level of proUGN in a biological sample of a subject, characterized in that a concentration level of proUGN of 2.241 ng/ml or higher indicates that the subject is positive for stage 3 or stage 4 eGFR-defined CKD.

The proUGN level (or amount or value) may be measured in a biological sample from the subject, such as a biological fluid sample or a biological tissue sample or an exosome. There are many well-known methods for measuring a biomarker, such as proUGN, from a biological sample. For example, measuring the concentration level of proUGN in a biological sample may be performed by an immunoassay such as enzyme linked immunosorbent assay (ELISA), counting immunoassay (CIA), chemiluminescence immunoassay (CLIA), electrochemiluminescence immunoassay (ECLIA), radioimmunoassay (RIA), immunoradiometric assay (IRMA) or western blot.

In a particular embodiment, the proUGN concentration level is measured using an immunoassay such as an ELISA. ELISAs are well-known to those skilled in art. In one example, an ELISA as described in U.S. Patent Publication No. 2021/0263047 (again incorporated by reference in its entirety) is used to measure the proUGN concentration of the subject.

In an additional embodiment, the method for diagnosing a subject with stage 3 or stage 4 eGFR-defined CKD can also include treating the subject with one or more CKD-therapeutic agents, such as those discussed above.

### D. Method of Diagnosing and/or Treating Pre-CKD

Early identification of patients with pre-CKD is important in order to perform early interventions and reduce progression to kidney failure and/or cardiovascular events. As such, the inventors have discovered that a proUGN concentration range that can be used to diagnose and/or treat a subject as having “pre-CKD,” where pre-CKD is as defined herein.

Thus, in another embodiment a method of treating pre-CKD in a subject is provided herein. The method comprises providing lifestyle and/or dietary change instructions to the subject in order to decrease or prevent their progression to CKD when the subject has a proUGN concentration level of 2.05 ng/ml to and including 2.24 ng/ml in a biological sample. Any appropriate biological sample from the subject and measurement technique, such as those described herein, can be used to detect and quantify the proUGN concentration level of the subject.

The range of proUGN concentration level of 2.05 ng/ml to and including 2.24 ng/ml, which is just before the CKD threshold value of 2.241 ng/ml, is important diagnostic information and indicates that the subject has pre-CKD and is at risk of progressing to CKD. The subject can be instructed to make various lifestyle and/or dietary changes in order to reduce or stop the progression to a CKD. Strategies include general lifestyle measures, which improve cardiovascular health, BP control, and interruption of the renin-angiotensin-aldosterone system (RAAS). Non-limiting examples of lifestyle and/or dietary modifications include:
achieving or maintaining a healthy weight (BMI 20 to 25), lowering salt intake to <90 mmol (<2 g) per day of sodium (corresponding to 5 g of sodium chloride), unless contraindicated,
restriction of sodium intake for children with CKD who have hypertension (systolic and/or diastolic blood pressure >95th percentile) or prehypertension (systolic and/or diastolic blood pressure >90th percentile and <95th percentile), following the age-based Recommended Daily Intake.
Supplemental free water and sodium supplements for children with CKD and polyuria to avoid chronic intravascular depletion and to promote optimal growth.
Dietary advice and information in the context of an education program, tailored to severity of CKD and the need to intervene on salt, phosphate, potassium, and protein intake where indicated.
Undertaking an exercise program compatible with cardiovascular health and tolerance, aiming for at least 30 minutes 5 times per week.
Smoking cessation.
Limiting alcohol intake to no more than two standard drinks per day for men and no more than one standard drink per day for women.
Avoiding high protein intake (41.3 g/kg/day).

In a further embodiment, a method of diagnosing pre-CKD in a subject is also provided herein. The method comprises measuring a concentration level of proUGN in a biological sample of the subject, wherein a concentration level of 2.05 ng/ml to and including 2.24 ng/ml indicates that the subject is positive for pre-CKD. As noted above, any appropriate biological sample from the subject and measurement technique, such as those described herein, can be used to detect and quantify the proUGN concentration level of the subject.

Additionally, the method of diagnosing pre-CKD by measuring the subject's proUGN concentration level in a biological sample may also include a “treatment” step, such as providing lifestyle and/or dietary change instructions, such as those described above, to the subject to decrease or prevent progression to CKD.

### E. Additional Biomarkers

In some embodiments, one or more biomarker in addition to proUGN can be tested along with proUGN. In certain embodiments, additional clinical indicia may be used in conjunction with proUGN alone, or fragments thereof, or in combination with other biomarker or clinical chemistry assay results wherein a combination of biomarker levels relevant to kidney health or dysfunction are measured which include but are not limited to serum creatinine (sCr), urine albumin, BUN, cystatin C, urea, neutrophil gelatinase (NGAL), kidney injury molecule-1 (KIM-1), L-fatty acid binding protein (L-FABP), FABP1, IGFBP-7 (insulin-like growth factor-binding protein-7), TIMP-2 (tissue inhibitor of metalloproteinases-2), calprotectin, IP-10 (interferon gamma-induced protein-10), MCP-1 (monocyte chemoattractant protein-1), Ins2 (insulin-2), LepR (leptin receptor), beta trace protein (lipocalin prostaglandin D2 synthase), ADMA (asymmetric dimethylarginine), SDMA, uromodulin (Tamm-Horsfall protein), NAG (N-acetyl-glucosaminidase), GST (glutathione-S-transferase), GGT (gamma-glutamyl transpeptidase), AAP (alanine aminopeptidase), LDH (lactate dehydrogenase), Gd-IgA1 (galactose-deficient IgA1), PLA2R M-type phospholipase A2 receptor, THSD7A (thrombospondin type 1 domain containing 7A), TMEM2 (transmembrane protein 2), polycystin-1, polycystin-2, hydroxyproline, oxalate, IL-6, IL-9, IL-17, IL-18, actin-alpha, podocin, FOXP3 (forkhead box-P3), MMP12, GATA-3, synaptopodin, alpha actinin-4, Wilms tumor-1, CCL14, T-box expressed in T-cells (T-bet), clusterin, B2 microglobulin, trefoil factor-3, osteopontin, podocalyxin, and renal papillary antigen (RPA-1). This embodiment includes a combination assay with high sensitivity and specificity for proUGN in addition to other relevant biomarkers, specifically, those mentioned herein.

In some embodiments, one or more biomarkers in addition to proUGN can be measured in order to treat CKD. In some embodiments, a method of treating stage 3 or stage 4 CKD may include administering a CKD-therapeutic agent to a subject in need thereof, where one or more biomarker in addition to proUGN is measured. In some embodiments, the proUGN concentration may be 2.241 ng/ml or higher in a biological sample of the subject. In some embodiments, the additional biomarker being measured is serum creatinine.

In some embodiments, one or more biomarkers in addition to proUGN can be measured in order to diagnose CKD. In some embodiments, a method of diagnosing stage 3 or stage 4 CKD may include measuring one or more biomarkers in addition to proUGN. In some embodiments, the proUGN concentration may be 2.241 ng/ml in a biological sample of the subject. In some embodiments, a proUGN concentration of 2.241 nl/ml or higher may indicate that the subject is positive for stage 3 or 4 CKD. In some embodiments, the additional biomarker being measured is serum creatinine.

In some embodiments, one or more biomarkers in addition to proUGN can be measured in order to treat pre-CKD. In some embodiments, a method of treating pre-CKD may include providing lifestyle and/or dietary modification instructions to the subject to decrease progression to CKD, where one or more biomarker in addition to proUGN is measured. In some embodiments, the proUGN concentration may be between 2.05 ng/ml to and including 2.24 ng/ml in a biological sample of the subject. In some embodiments, the additional biomarker being measured is serum creatinine.

In some embodiments, one or more biomarkers in addition to proUGN can be measured in order to diagnose pre-CKD. In some embodiments, a method of diagnosing pre-CKD may include measuring one or more biomarkers in addition to proUGN. In some embodiments, the proUGN concentration may be between 2.05 ng/ml to and including 2.24 ng/ml in a biological sample of the subject. In some embodiments, a proUGN concentration between 2.05 ng/ml to and including 2.24 ng/ml may indicate that the subject is positive for pre-CKD. In some embodiments, the additional biomarker being measured is serum creatinine.

Similar to proUGN, measuring the concentration level of one or more additional biomarkers in a biological sample may be performed by an immunoassay such as enzyme linked immunosorbent assay (ELISA), counting immunoassay (CIA), chemiluminescence immunoassay (CLIA), electrochemiluminescence immunoassay (ECLIA), radioimmunoassay (RIA), immunoradiometric assay (IRMA) or western blot.

### EXAMPLES

The following examples are merely illustrative, and do not limit this disclosure in any way.

### Example 1 Validation Study

A phase 2 validation study was performed to evaluate the biomarker, prouroguanylin (proUGN), for evaluation of kidney function, detection, or diagnosis, and/or management of kidney disease using the immunological assay (ELISA) disclosed in U.S. Patent Publication No. 2021/0263047, again incorporated by reference in its entirety.

The validation study presented herein entails 384 patients, whereby 137 patients in the study were clinically confirmed by standard of care for chronic kidney disease (CKD) and staged according to the respective patient's estimated GFR (eGFR) baseline, employing the race-free 2021 CKD-EPI eGFR equation. Of the 137 patients defined as having CKD, 123 patients were staged 3 and 4. There were 247 patients included in this study that were determined to have renal function within normal limits by eGFR and no prior diagnosis of CKD. While CKD diagnosis by the physician is the primary outcome, comorbidities, risk factors and known etiologies were recorded for both the cases and controls; although, many controls did not have any listed diseases or conditions reported. For controls without disease (n=107), risk factors or known CKD etiologies, serum was prepared from a venous blood draw as part of their basic metabolic panel (BMP) or comprehensive metabolic panel (CMP) testing during a semi-annual or annual examination. Demographics such as age, sex, race, and ethnicity were included in the patient data reports and closely represented the current patient percentage or rate of the respective demographic category within the U.S. with or without CKD as defined by the CDC and U.S. Renal Data System (www.cdc.gov/kidneydisease/publications-resources/CKD-national-facts.html, accessed Dec. 15, 2023). For example, African Americans make up approximately 13% of the U.S. population but account for approximately 31% of patients with CKD. Also, women are more prone to CKD than men—˜14% and ˜12%, respectively. This validation study represents such demographics. Serum creatinine values and other biomarkers were validated in 10% of the study samples to ensure reproducibility and authenticity of the data elements. Medications and ICD-10 coded comorbidities were also recorded for subjects at the time of sample collection. Comorbidities, etiologies, and/or risk factors of CKD included: hypertension (42%), diabetes (24%), congestive heart failure (CHF) (9%), obesity (21%), history of acute kidney injury (3%), and other cardiovascular disease risk factors (10%). Recorded medications included: angiotensin-converting enzyme inhibitors (ACEi), angiotensin-receptor blockers (ARB), diuretics, calcium channel blockers, beta blockers, statins, and metformin. Exclusion criteria consisted of patients/subjects <18 years old, actively septic, pregnant, or diagnosed with Stage 1 or Stage 2 CKD. For Cases, patients who had a reported eGFR baseline <60 mL/min/1.73 m2 and associated ICD10 diagnosis code for Stage 3a, 3b, 4, or 5 were included. For controls, renal function was considered within normal limits if cGFR was calculated to be >60 mL/min/1.73 m2 by the CKD-EPI creatinine-based eGFR (2021) and the patient/subject had no history of CKD. A nested evaluation of healthy control subjects (n=48) also had their mGFR determined via iohexol plasma clearance employing a multi-sampling protocol. All subjects with iohexol-determined mGFR were without CKD and without history or presence of any risk factor or etiology of CKD, history of obesity, notable or remarkable cardiovascular disease (CVD), cancer, gastrointestinal or liver diseases, high blood pressure (BP), or diabetes.

### Definition of CKD Stages and Outcome Parameters

Subjects were assigned to one of four stages of CKD (G3a, G3b, 4, or 5) based on patient medical history files and reported baseline eGFR value. Serum creatinine levels were independently utilized to determine the CKD-EPI (2021) eGFR for each patient sample, which was used in the data analysis. Patients were evaluated in the study based on their reported stage if the calculated eGFR_2021 value shifted their stage by ≤1-degree. No patients eGFR differed from reported stage by more than one degree. We included 247 randomly selected patients with an eGFR value within normal limits (≥60 ml/min per 1.73 m²), as determined by the CKD-EPI (2021) equation, and no prior history of CKD, including stages 1 or 2.

Clinical diagnosis of CKD is defined when a patient has two low eGFR values >90-days apart, and staged with an established eGFR baseline in accordance to Kidney Disease Improving Global Outcomes (KDIGO) guidelines: G3a is defined as having a baseline eGFR <60 ml/min/1.73 m² and ≥45 ml/min/1.73 m²; G3b is defined as having a baseline eGFR <45 ml/min/1.73 m² and ≥30 ml/min/1.73 m²; G4 is defined as having a baseline eGFR <30 ml/min/1.73 m² and ≥15 ml/min/1.73 m²; and G5 is defined as having a baseline eGFR <15 ml/min/1.73 m². The primary outcome of the study was quantification of proUGN in patient serum samples. One serum sample was collected from each subject for testing. The secondary outcomes were diagnosis of CKD or confirmation of no CKD via physician/patient medical reports, sCr levels, and eGFR. Serum creatinine and eGFR were reported in all physician/patient medical records.

### Sample Collection

Serum from overnight fasted patients were prepared from venipuncture blood collection using BD Vacutainer™ Venous Blood Collection SST™ Serum Separation Tubes (Becton Dickinson, Franklin Lakes, NJ). Once collected, tubes were gently inverted 5-8 times to mix the clot activator with the blood and allowed to clot for 30-45 min at RT. Tubes were then centrifuged at 1300×g for 10-15 min and serum frozen in 1 mL aliquots at −80° C. and shipped on dry ice. Hemolyzed, icteric, or lipemic samples were rejected. Biomarkers were measured on serum samples (stored at −80° C.) from sample aliquots of three or less freeze-thaw cycles.

### Biomarker Testing

Serum creatinine was reported for all patients using a routine creatinine analysis performed on an automated analyzer, employing the Jaffe's reaction method and standard clinical chemistry IDMS traceability and standardization. To address, in part, rigor within the analytical variation and ensure reproducibility and authenticity of the data elements from the sCr reported measurements, the Cincinnati Biomarker Laboratory (Cincinnati Children's Hospital; Cincinnati, OH) randomly tested the sCr levels in 10% of our cohort in a blind format. Validation of the reported creatinine values against 3^{rd} party evaluation was ±3.1% coefficient of variation. Estimated GFR was determined employing the race-free 2021 CKD-EPI sCr-based equation without race as a bias.

Measurement of Circulating proUGN

Circulating proUGN was quantitatively measured via a mAb sandwich ELISA. Monoclonal antibodies and a stably-expressed human cell line was developed to express human proUGN, without the addition of any tags or conjugates, employing previously described methods. The proUGN calibrator protein was confirmed by 2D-gel electrophoresis followed by Western blotting and protein identification by MALDI-TOF/TOF (Applied Biomics; Hayward, CA) and independently by a multiple reaction monitoring LC-MS/MS of endonuclease digested samples followed by targeted peptide sequencing (Peptides International/BioSynth (Louisville, KY); Alphalyse (Palo Alto, CA)). The MRM LC-MS/MS method was also used to confirm the concentration of proUGN in human serum samples as an independent method of measurement of the biomarker. To determine the concentration of proUGN in a serum sample, a standard curve was constructed with the calibrator proUGN by plotting absorbance values vs. known proUGN concentrations of standards. Quantitative results were calculated with a robust linear regression model (log-transformed, 5-parameter curve fit). The mAb-based sandwich ELISA for circulating proUGN has a limit of detection of 2.0 pg/mL, detection range of 20 pg/mL to 2000 pg/ml, intra-assay precision of <3.0% and intra-assay precision of <4.9%. The assay has high specificity for detection of proUGN in human serum with no cross-reactivity or interference between structurally similar analogs or guanylate cyclase activator proteins. Each serum sample measured for circulating proUGN was performed in duplicate or triplicate and results presented are representative of the mean of 3-6 experiments±standard error of the mean (SEM).

Table 1 immediately below is the serum prouroguanylin (proUGN) levels by CKD Stage using the immunological assay (ELISA) disclosed in U.S. Patent Publication No. 2021/0263047. The number of subjects per stage are shown below for this validation study.

Prouroguanylin (proUGN)

| CKD | | | Average | Standard | | Coefficient | Range |
|---|---|---|---|---|---|---|---|
| Stage | Gender | n= | (ng/mL) | Deviation | SEM | of Variation | (min-max) |
| Stage 5 | Combined | 14 | 14.619 | 6.292 | 1.682 | 43.0% | |
| | Male | 3 | 15.584 | 6.304 | 3.640 | 40.5% | 9.47-22.06 |
| | Female | 11 | 14.356 | 6.570 | 1.981 | 45.8% | 5.69-22.06 |
| Stage 4 | Combined | 31 | 5.806 | 2.641 | 0.474 | 45.5% | |
| | Male | 17 | 5.996 | 2.656 | 0.644 | 44.3% | 3.04-12.81 |
| | Female | 14 | 5.576 | 2.703 | 0.722 | 48.5% | 2.88-12.79 |
| Stage 3b | Combined | 66 | 4.008 | 2.420 | 0.298 | 60.4% | |
| | Male | 36 | 4.617 | 3.072 | 0.512 | 66.5% | 2.00-16.83 |
| | Female | 30 | 3.278 | 0.852 | 0.156 | 26.0% | 2.25-5.26 |
| Stage 3a | Combined | 26 | 3.961 | 3.261 | 0.640 | 82.3% | |
| | Male | 22 | 3.704 | 3.129 | 0.667 | 84.5% | 1.70-17.25 |
| | Female | 4 | 5.372 | 4.117 | 2.059 | 76.6% | 2.78-11.51 |
| No CKD* | Combined | 239 | 1.105 | 0.480 | 0.031 | 43.5% | |
| | Male | 117 | 1.203 | 0.491 | 0.045 | 40.8% | 0.35-2.24 |
| | Female | 122 | 1.010 | 0.452 | 0.041 | 44.7% | 0.34-2.18 |

*False positives not included (n = 8)

Table 2 immediately below is GFR per CKD Stage. A summary of GFR for patients with normal renal function and patients clinically diagnosed and staged for CKD. The number of subjects per stage is shown below for this validation study.

Glomerular Filtration Rate (GFR)

| CKD | | | Average | Standard | | Coefficient | Range |
|---|---|---|---|---|---|---|---|
| Stage | Gender | n= | (ml/min/1.7 m²) | Deviation | SEM | of Variation | (min-max) |
| Stage 5 | Combined | 14 | 12.1 | 6.6 | 1.8 | 54.5% | |
| | Male | 3 | 12.3 | 5.7 | 3.3 | 46.2% | 6-25 |
| | Female | 11 | 12.1 | 11.0 | 3.3 | 91.0% | 4-26 |
| Stage 4 | Combined | 31 | 22.5 | 5.1 | 0.9 | 22.8% | |
| | Male | 17 | 22.3 | 5.8 | 1.4 | 26.2% | 12-34 |
| | Female | 14 | 22.9 | 4.7 | 1.3 | 20.6% | 14-31 |
| Stage 3b | Combined | 66 | 35.6 | 5.5 | 0.7 | 15.5% | |
| | Male | 36 | 35.8 | 6.4 | 1.1 | 17.9% | 25-50 |
| | Female | 30 | 35.3 | 4.3 | 0.8 | 12.1% | 29-42 |
| Stage 3a | Combined | 26 | 48.8 | 4.7 | 0.9 | 9.6% | |
| | Male | 22 | 48.7 | 4.9 | 1.0 | 10.1% | 43-59 |
| | Female | 4 | 49.2 | 3.8 | 1.9 | 7.7% | 45-54 |
| No CKD* | Combined | 239 | 106.8 | 27.0 | 1.7 | 25.3% | |
| | Male | 117 | 105.9 | 28.7 | 2.7 | 27.1% | 60-206 |
| | Female | 122 | 107.6 | 25.4 | 2.3 | 23.6% | 60-173 |

*False positives not included (n = 8)

Table 3 immediately below shows the demographics of subjects in the Normal/Healthy population per serum proUGN levels in this validation study.

| | n= | Average | SEM | Median |
|---|---|---|---|---|
| Control - Male | 117 | 1.203 | 0.038 | 1.150 |
| Race/Ethnicity | | | | |
| Non-Hispanic White | 83 | 1.268 | 0.056 | 1.218 |
| Non-Hispanic Black | 14 | 1.288 | 0.110 | 1.329 |
| Hispanic or Latino | 10 | 0.936 | 0.100 | 0.834 |
| Non-Hispanic Asian | 3 | 1.327 | 0.491 | 1.194 |
| Control - Female | 122 | 1.010 | 0.041 | 0.940 |
| Race/Ethnicity | | | | |
| Non-Hispanic White | 62 | 1.046 | 0.065 | 1.035 |
| Non-Hispanic Black | 24 | 1.015 | 0.080 | 0.929 |
| Hispanic or Latino | 15 | 1.097 | 0.073 | 1.054 |
| Non-Hispanic Asian | 3 | 1.068 | 0.155 | 0.965 |
| Control - Total | 239 | 1.105 | 0.031 | 1.054 |

Levels were significantly increased in the Control-Male (n=117, Avg. 1.20 ng/ml) group compared to the Control-Female (n=122, Avg. 1.01 ng/ml) population (p<0.002). No significant difference was seen between Race/Ethnicity subpopulations (shown above in Table 3, p>0.10 for all compared subpopulations). Although differences in the mean was observed in gender, the clinical threshold value that stratifies patients to begin medical intervention for eGFR-defined CKD is not affected, and remains 2.241 ng/ml. The reference range defined by the 95% reference interval (95RI) for the Control—Total population exhibited a lower reference limit (LRL), or lower limit of normal (0.162, 90% CI 0.11-0.22 ng/ml) and upper reference limit (URL), or upper limit of normal (2.049, 90% CI 2.00-2.10 ng/ml). The values between the URL and the cut-off threshold value (2.05-2.24 ng/ml) is defined as pre-CKD, and indicates the subject should be provided lifestyle and/or dietary change instructions to decrease progression to CKD.

Table 4 immediately below is the average serum creatinine values for subjects per stage of CKD in this validation study.

Serum Creatinine (sCr)

| CKD | | | Average | Standard | | Coefficient | Range |
|---|---|---|---|---|---|---|---|
| Stage | Gender | n= | (mg/dl) | Deviation | SEM | of Variation | (min-max) |
| Stage 5 | Combined | 14 | 5.35 | 2.24 | 0.60 | 41.8% | |
| | Male | 3 | 6.75 | 3.47 | 2.00 | 51.4% | 2.75-8.90 |
| | Female | 11 | 4.97 | 1.83 | 0.55 | 36.9% | 2.35-9.29 |
| Stage 4 | Combined | 31 | 2.69 | 0.66 | 0.12 | 24.7% | |
| | Male | 17 | 3.03 | 0.66 | 0.16 | 21.6% | 2.10-4.40 |
| | Female | 14 | 2.28 | 0.39 | 0.10 | 17.1% | 1.60-2.80 |
| Stage 3b | Combined | 66 | 1.80 | 0.31 | 0.04 | 17.2% | |
| | Male | 36 | 1.97 | 0.30 | 0.05 | 15.2% | 1.40-2.70 |
| | Female | 30 | 1.59 | 0.16 | 0.03 | 9.8% | 1.40-2.03 |
| Stage 3a | Combined | 26 | 1.48 | 0.16 | 0.03 | 10.5% | |
| | Male | 22 | 1.51 | 0.15 | 0.03 | 10.0% | 1.30-1.80 |
| | Female | 4 | 1.34 | 0.08 | 0.04 | 5.9% | 1.24-1.40 |
| No CKD* | Combined | 239 | 0.83 | 0.23 | 0.01 | 27.7% | |
| | Male | 117 | 0.95 | 0.22 | 0.02 | 23.4% | 0.37-1.41 |
| | Female | 122 | 0.72 | 0.18 | 0.02 | 24.3% | 0.36-1.20 |

*False positives not included (n = 8)

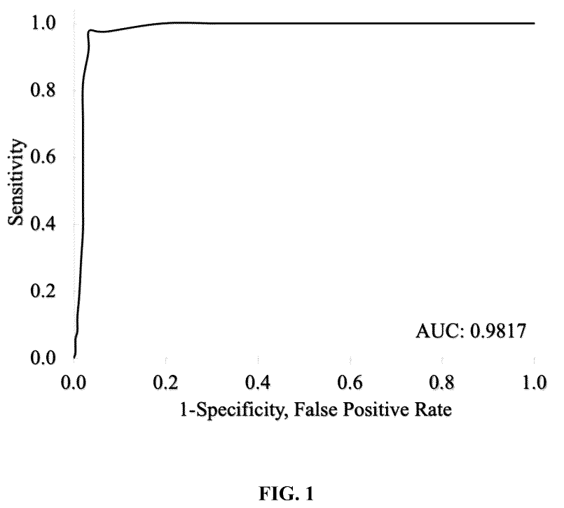

FIG. 1 disclosed herein shows a receiver-operator curve (ROC) analysis was employed to determine the specificity (96.8%) and sensitivity (97.6%) to discriminate between eGFR-defined CKD subjects, whom were clinically diagnosed by a practicing physician, and controls with no prior history of chronic kidney disease and eGFR values within normal limits. The area under the curve (AUC) for the discrimination between controls and eGFR-defined CKD is 0.9817 with a cut-off threshold value 2.241 ng/ml.

eGFR-defined CKD subjects (n=123) were significantly stratified for appropriate medical intervention against a healthy control population (n=247). There were 3 false-negatives, i.e., patients with eGFR-defined CKD who were below the clinical threshold value. Overall, there were 8 false-positives, or subjects with an eGFR within normal limits and without CKD and were above the clinical threshold. The ROC of this data determined sensitivity (97.8%), specificity (96.8%), positive predictive value (PPV) (94.4%), and NPV (98.8%), and an AUC of 0.982 (Table 5).

TABLE 5
ROC analysis of serum proUGN levels for CKD vs Controls

| | CKD+ (G3-G5) | Controls | |
|---|---|---|---|
| ProUGN(+) | 134 | 8 | PPV: |
| >2.24 ng/ml | | | 94.4% |
| ProUGN(−) | 3 | 239 | NPV: |
| ≤2.24 ng/ml | | | 98.8% |
| | Sensitivity: | Specificity: | |
| | 97.8% | 96.8% | |

Clinical performance of serum proUGN to distinguish non-CKD from CKD patients who have been clinically diagnosed and classified into either stage 3a, 3b, 4, or 5. Utilizing the cut-off 2.24 ng/ml, serum proUGN was 97.8% sensitive and 96.8% specific for CKD in the 384-patient study. PPV was 94.4%, or likelihood that a positive test result ([proUGN]>2.24 ng/ml) correctly aligns with disease. NPV was 98.8%, or likelihood that a negative test result correctly reflects a non-disease control with normal renal function.

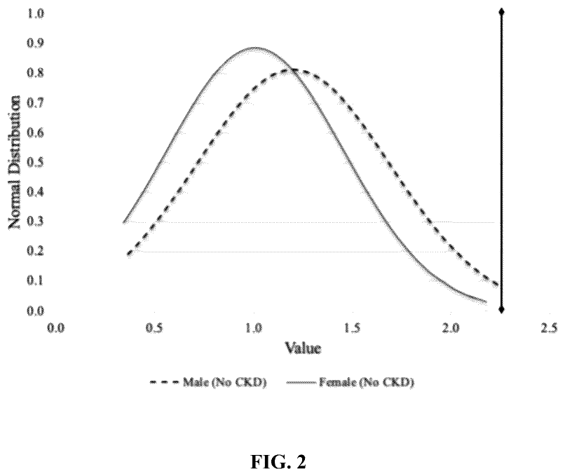

The overall accuracy to identify and stratify the eGFR-defined CKD population for immediate medical intervention against healthy controls was determined by ROC parameters to be 97.0%, specifically at the clinical threshold value 2.241 ng/ml. FIG. 2 shows normal gaussian distribution curves for serum proUGN (ng/ml) in healthy control subjects without a history of CKD and renal function within normal limits in relation to eGFR as defined by the National Kidney Foundation (NKF). When below the clinical cut-off threshold value, 2.241 ng/ml, that defines eGFR-defined CKD stratification (illustrated by the vertical black line), hormone levels demonstrate a normal symmetrical bell-shaped distribution centered around the mean. Despite the slight difference in the mean values between male and female control subjects (see Table 3), the eGFR-defined CKD cut-off threshold is unaffected and remains a congruent implication to begin medical treatment regardless of gender, race/ethnicity. Values above the clinical cut-off do not follow the natural biological law of analyte distribution within a population, and therefore ascertains the need to begin medical management to slow/prevent further progression.

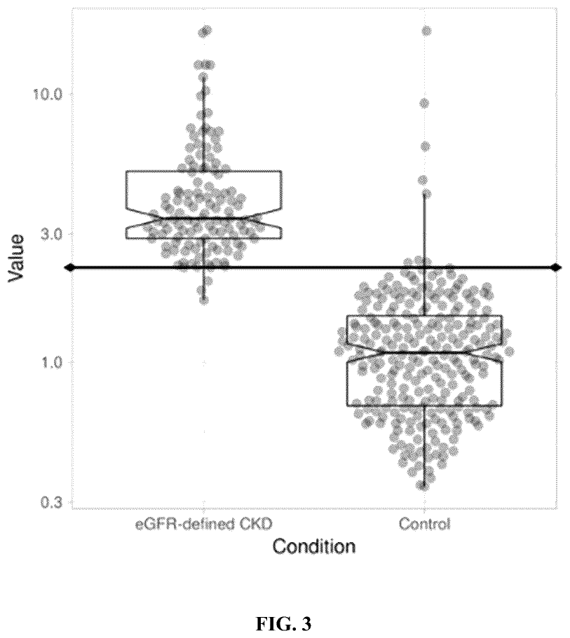

FIG. 3 shows levels of proUGN (ng/ml) in human subjects. Patient groups were stratified using the method of classification characterized by the National Kidney Foundation (NKF) as eGFR-defined CKD, having a baseline eGFR (at least two consecutive values at least three months apart) less than 60 ml/min/1.73 m², and compared against a control population with healthy renal function defined by an eGFR of at least equal to or greater than 60 ml/min/1.73 m².

The results of the subjects were blindly evaluated against the clinical cut-off threshold value, 2.241 ng/ml, represented by the dark horizontal line. eGFR-defined CKD subjects (n=123) were significantly stratified for appropriate medical intervention against a healthy control population (n=247) without CKD (p<0.0001). The results are shown in Table 6 below (SEM, Standard Error of the Mean). Table 6:

| | | | | | 95CI | | |
|---|---|---|---|---|---|---|---|
| | n= | Average | SEM | Median | median | LRL | URL |
| Control | 247 | 1.266 | 0.084 | 1.081 | 0.99-1.17 | 0.162 | 2.049 |
| eGFR- | 123 | 4.452 | 0.249 | 3.420 | 3.24-3.86 | | |
| defined | | | | | | | |
| CKD | | | | | | | |

FIG. 3 illustrates a traditional boxplot overlay for each subpopulation. They display variation in samples of a statistical population without making assumptions of the underlying statistical distribution, and indicate the degree of skewness or dispersion (spread) of the data. The central or middle line represents the median, while the lower line indicates first quartile value (Q1) and upper line the third quartile value (Q3). The interquartile range (IQR) is calculated by subtracting Q1 from Q3, and demonstrates the distance between the 25^{th} and 75th percentiles. Those values for each group are in Table 7:

| | IQR | Q1 | Q3 |
|---|---|---|---|
| Control | 0.80 | 0.68 | 1.48 |
| eGFR-defined CKD | 2.26 | 2.88 | 5.14 |

The lower reference limit (LRL), or the lower limit of normal, and upper reference limit (URL), or upper limit of normal, is defined as the 95% Reference Interval (RI) for a hormone within a normal healthy population. The URL is 2.049 ng/ml (90% CI, 1.996-2.100), indicating that subjects ≥2.05 ng/ml exceed the 97.5^{th} percentile, and thus, should be classified as pre-CKD, and should be provided with lifestyle and/or dietary change instructions to decrease progression to CKD. The normal reference range for the invention is shown above in Table 6.

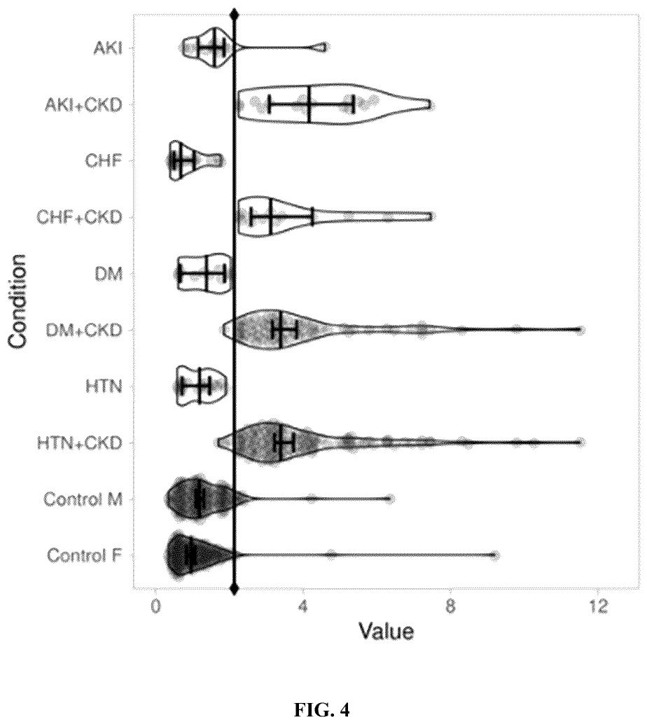

FIG. 4 shows serum proUGN level (ng/ml) in human subjects stratified into populations based on the presence of high-risk comorbid target conditions for developing CKD including: history of acute kidney injury (AKI), congestive heart failure (CHF), diabetes mellitus (DM), and hypertension (HTN); and further classified into subpopulations based on whether the subject has been previously established by a practicing physician to have eGFR-defined CKD. Otherwise the patient was determined by eGFR and UACR (urine-albumin creatinine ratio) to have normal, healthy renal function at the time of testing. Results are shown in Table 8:

| Condition | n= | Average | SEM | Median | 95CI median |
|---|---|---|---|---|---|
| AKI | 11 | 1.72 | 0.32 | 1.59 | 1.15-1.85 |
| AKI + CKD | 16 | 4.34 | 0.38 | 4.16 | 3.08-5.36 |
| CHF | 16 | 0.85 | 0.12 | 0.67 | 0.49-1.04 |
| CHF + CKD | 13 | 3.70 | 0.48 | 3.12 | 2.58-4.25 |
| DM | 10 | 1.33 | 0.19 | 1.38 | 0.66-1.87 |
| DM + CKD | 71 | 4.44 | 0.34 | 3.42 | 3.18-3.86 |
| HTN | 16 | 1.14 | 0.12 | 1.19 | 0.71-1.46 |
| HTN + CKD | 120 | 4.45 | 0.26 | 3.42 | 3.29-3.88 |
| Control M | 122 | 1.30 | 0.07 | 1.18 | 1.09-1.32 |
| Control F | 125 | 1.23 | 0.15 | 0.96 | 0.83-1.06 |

FIG. 4. illustrates a traditional boxplot overlay for each subpopulation. The central line represents the median, while the left line indicates Q1—i.e., the lower 25^{th} percentile of the dataset—the right line indicates Q3—the 75^{th} percentile of the dataset. The IQR, or the value at Q1 subtracted from Q3, demonstrates the range of the middle 50^{th} percentile with each given subpopulation. Those values for each group are in Table 9:

| Condition | IQR | Q1 | Q3 |
|---|---|---|---|
| AKI | 0.52 | 1.25 | 1.77 |
| AKI + CKD | 2.29 | 3.03 | 5.32 |
| CHF | 0.57 | 0.48 | 1.05 |
| CHF + CKD | 1.67 | 2.58 | 4.25 |
| DM | 1.01 | 0.76 | 1.77 |
| DM + CKD | 1.75 | 2.84 | 4.58 |
| HTN | 0.79 | 0.70 | 1.49 |
| HTN + CKD | 2.24 | 2.88 | 5.12 |
| Control M | 0.93 | 0.79 | 1.72 |
| Control F | 0.73 | 0.62 | 1.36 |

### Example 2 Validation Study-Additional Findings in CKD-Related Comorbidities

Following the same phase 2 validation study described in Example 1, the circulating proUGN was measured in patients of high-risk of CKD; i.e., patients with comorbidities commonly associated with CKD. Slightly more individuals are included in these additional findings because an AI based search algorithm was used to sift through hundreds of pages of patient data records and reports, and a few more individuals included in the study were found to be diagnosed with these comorbidities, although they were not coded correctly into their charts.

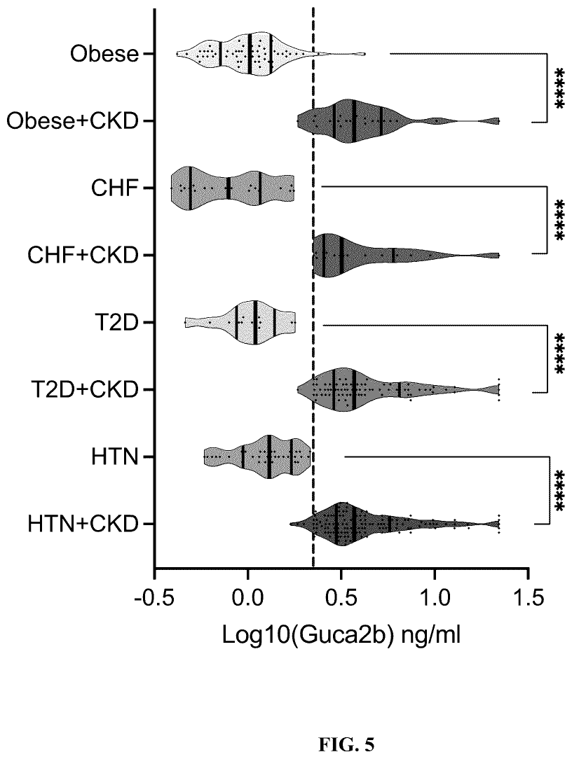

Nested case-controls for this study included patients diagnosed with both CKD and the comorbidity. Results demonstrate that clinical samples from patients with comorbidities of hypertension (HTN), type 2 diabetes (T2D), congestive heart failure (CHF) or obesity without CKD were significantly lower than patients with both the identified comorbidity patient who was diagnosed with CKD. FIG. 5 shows the following disease-controls vs cases: Hypertension (n): 1.31±0.08 (34) vs 5.32±0.38 (129), P<0.0001; TD2 (n): 1.11±0.10 (14) vs 5.65±0.56 (79), P<0.0001; CHF (n): 0.91±0.10 (20) vs 5.13±1.24 (16), P<0.0001; Obesity (n), 1.10±0.07 (61) vs 4.91±0.95 (21), P<0.0001). Table 10 provides a summary of the statistical results from each patient population with and without CKD including the median with the ICR (95% Cl). Between-group differences were analyzed with an unpaired two-tailed Students t-tests. proUGN levels in non-CKD male patients are higher than female controls (*P<0.05), and controls are significantly lower than G3a regardless of gender (****P<0.0001). G3b is significantly lower than G4 (****P<0.0001), and are not significantly different than G3a (ns, P=0.54). Levels in G5 are significantly higher than G4 (****P<0.0001). Black lines within grey violin distribution indicate the median, the 25th and 75th quartile. The dashed vertical line represents the 2.241 ng/ml threshold for CKD. See Table 10 for data analysis.

TABLE 10
Comparison of serum proUGN levels by presence of
comorbid disease in CKD and Control populations.

| | HTN + | | T2D + | | CHF + | | Obese + | |
|---|---|---|---|---|---|---|---|---|
| | CKD | HTN | CKD | T2D | CKD | CHF | CKD | Obese |
| n= | 129 | 34 | 79 | 14 | 16 | 20 | 21 | 61 |
| Median | 3.70 | 1.30 | 3.70 | 1.10 | 3.18 | 0.79 | 3.70 | 1.03 |
| 25th | 2.97 | 0.94 | 2.88 | 0.87 | 2.55 | 0.49 | 2.90 | 0.71 |
| quartile | | | | | | | | |
| 75th | 5.74 | 1.71 | 6.47 | 1.39 | 6.04 | 1.16 | 5.18 | 1.33 |
| quartile | | | | | | | | |
| 95% CI: | | | | | | | | |
| Lower | 3.34 | 1.06 | 3.33 | 0.795 | 2.54 | 0.512 | 3.05 | 0.895 |
| limit | | | | | | | | |
| Upper | 4.1 | 1.56 | 4.25 | 1.39 | 6.3 | 1.15 | 4.99 | 1.15 |
| limit | | | | | | | | |
| Mean | 5.32 | 1.31 | 5.65 | 1.11 | 5.13 | 0.91 | 4.91 | 1.10 |
| SEM | 0.38 | 0.08 | 0.56 | 0.10 | 1.24 | 0.10 | 0.95 | 0.07 |
| 95% CI: | | | | | | | | |
| Lower | 4.57 | 1.16 | 4.54 | 0.89 | 2.48 | 0.69 | 2.94 | 0.96 |
| limit | | | | | | | | |
| Upper | 6.08 | 1.47 | 6.76 | 1.32 | 7.79 | 1.13 | 6.89 | 1.25 |
| limit | | | | | | | | |

| | | | | |
|---|---|---|---|---|
| P-value | ****<0.0001 | ****<0.0001 | ****<0.0001 | ****<0.0001 |

Serum proUGN levels are shown based on the comorbid presence of a CKD-related condition and grouped into non-CKD and clinically diagnosed CKD populations to evaluate for significance. Comorbidity assessment demonstrated significantly elevated levels of proUGN only when CKD was present: hypertension (HTN) with and without CKD (****P<0.0001), type 2 diabetes (T2D) with and without CKD (****P<0.0001), congestive heart failure (CHF) with and without CKD (****P<0.0001), and obesity with and without CKD (****P<0.0001). The 95% CI (95% Confidence Interval) upper and lower limits are reported for the mean and median of each group, as well as the interquartile range (IQR, 25^{th} to 75^{th} quartiles) and SEM (Standard Error of the Mean). All references, including publications, patent applications, and patents, cited herein are hereby incorporated by reference to the same extent as if each reference were individually and specifically indicated to be incorporated by reference and were set forth in its entirety herein.

Particular embodiments of this invention are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of treating stage 3 or stage 4 chronic kidney disease (CKD), the method comprising administering a CKD-therapeutic agent to a subject in need thereof, wherein the subject has a proUGN concentration level of 2.241 ng/ml or higher in a biological sample of the subject.

2. The method of claim 1, wherein the subject has acute kidney injury.

3. The method of claim 1, wherein the subject was diagnosed with COVID-19 about 12 months or less prior to a proUGN concentration level of 2.241 ng/ml or higher in the biological sample.

4. The method of claim 1, wherein the subject has diabetes.

5. The method of claim 1, wherein the subject has hypertension.

6. The method of claim 1, wherein the subject has drug-induced nephrotoxicity.

7. The method of claim 1, wherein the subject has clinically-diagnosed obesity.

8. The method of claim 1, wherein the CKD-therapeutic agent is a sodium-glucose cotransporter-2 (SGLT2) inhibitor; a potassium-sparing diuretic/selective mineralocorticoid-receptor antagonist; a thiazolidinedione; or an antifibrotic/pyridine.

9. The method of claim 8, wherein the CKD-therapeutic agent is a sodium-glucose cotransporter-2 (SGLT2) inhibitor.

10. The method of claim 8, wherein the CKD-therapeutic agent is a potassium-sparing diuretic/selective mineralocorticoid-receptor antagonist.

11. The method of claim 8, wherein the CKD-therapeutic agent is a thiazolidinedione.

12. The method of claim 8, where the CKD-therapeutic agent is an antifibrotic/pyridine.

13. The method of claim 1, wherein the biological sample is a biological fluid sample or a biological tissue sample or an exosome.

14. The method of claim 13, wherein the biological sample is a biological fluid sample comprising serum, blood, urine, plasma, saliva, cerebrospinal fluid, amniotic fluid, lung lavage, nasal lavage, gastrointestinal fluid, or interstitial fluid.

15. The method of claim 1, wherein the proUGN is full length proUGN (SEQ ID NO: 1), or a fragment thereof, or a variant thereof.

16. The method of claim 1, comprising measuring one or more biomarkers in addition to proUGN.

17. The method of claim 16, wherein the biomarker is serum creatinine.

18. A method for diagnosing stage 3 or stage 4 CKD in a subject, the method comprising measuring a concentration level of proUGN in a biological sample of the subject, characterized in that a concentration level of proUGN of 2.241 ng/ml or higher indicates that the subject is positive for stage 3 or stage 4 CKD.

19. The method of claim 18, wherein the biological sample is a biological fluid sample or a biological tissue sample or an exosome.

20. The method of claim 18, wherein measuring the concentration level of proUGN in the biological sample is performed by an immunoassay.

21. The method of claim 18, further comprising treating the subject with a CKD-therapeutic agent.

22. The method of claim 18, comprising measuring one or more biomarkers in addition to proUGN.

23. The method of claim 22, wherein the biomarker is serum creatinine.

24. A method of treating pre-CKD in a subject in need thereof, the method comprising providing lifestyle and/or dietary modification instructions to the subject to decrease progression to CKD, wherein the subject has a proUGN concentration level of 2.05 ng/ml to and including 2.24 ng/ml in a biological sample of the subject.

25. The method of claim 24, wherein the biological sample is a biological fluid sample or a biological tissue sample or an exosome.

26. The method of claim 24, comprising measuring one or more biomarkers in addition to proUGN.

27. The method of claim 26, wherein the biomarker is serum creatinine.

28. A method for diagnosing pre-CKD in a subject, the method comprising measuring a concentration level of proUGN in a biological sample of the subject, characterized in that a concentration level of 2.05 ng/ml to and including 2.24 ng/ml indicates that the subject is positive for pre-CKD.

29. The method of claim 28, wherein the measuring the concentration level of proUGN in the biological fluid sample is performed by an immunoassay.

30. The method of claim 28, further comprising providing lifestyle and/or dietary modification instructions to the subject to decrease progression to CKD.

31. The method of claim 28, comprising measuring one or more biomarkers in addition to proUGN.

32. The method of claim 31, wherein the biomarker is serum creatinine.


SEQUENCE LISTING (4 sequences)

SEQ ID NO: 1 - protein, 86 aa, Homo sapiens
VYIQYQGFRVQLESMKKLSDLEAQWAPSPRLQAQSLLPAVCHHPALPQDLQPVCASQEASSIFKTLRTIANDDCELCVNVACTGCL

SEQ ID NO: 2 - protein, 70 aa, Homo sapiens
VYIQYQGFRVQLESMKKLSDLEAQWAPSPRLQAQSLLPAVCHHPALPQDLQPVCASQEASSIFKTLRTIA

SEQ ID NO: 3 - protein, 22 aa, Homo sapiens
DLEAQWAPSPRLQAQSLLPAVC

SEQ ID NO: 4 - protein, 16 aa, Homo sapiens
NDDCELCVNVACTGCL